SEQUENCE LISTING (138 sequences)

SEQ ID NO: 1 - protein, 74 aa, Homo sapiens
GPASVPTTCCFNLANRKIPLQRLESYRRITSGKCPQKAVIFKTKLAKDICADPKKKWVQDSMKYLDQKSPTPKP

SEQ ID NO: 2 - protein, 6 aa, Artificial
  Eotaxin fragment
GPASVP

SEQ ID NO: 3 - protein, 7 aa, Artificial
  Eotaxin fragment
GPASVPT

SEQ ID NO: 4 - protein, 8 aa, Artificial
  Eotaxin fragment
GPASVPTT

SEQ ID NO: 5 - protein, 9 aa, Artificial
  Eotaxin fragment
GPASVPTTC

SEQ ID NO: 6 - protein, 10 aa, Artificial
  Eotaxin fragment
GPASVPTTCC

SEQ ID NO: 7 - protein, 11 aa, Artificial
  Eotaxin fragment
GPASVPTTCCF

SEQ ID NO: 8 - protein, 12 aa, Artificial
  Eotaxin fragment
GPASVPTTCCFN

SEQ ID NO: 9 - protein, 13 aa, Artificial
  Eotaxin fragment
GPASVPTTCCFNL

SEQ ID NO: 10 - protein, 14 aa, Artificial
  Eotaxin fragment
GPASVPTTCCFNLA

SEQ ID NO: 11 - protein, 15 aa, Artificial
  Eotaxin fragment
GPASVPTTCCFNLAN

SEQ ID NO: 12 - protein, 16 aa, Artificial
  Eotaxin fragment
GPASVPTTCCFNLANR

SEQ ID NO: 13 - protein, 17 aa, Artificial
  Eotaxin fragment
GPASVPTTCCFNLANRK

SEQ ID NO: 14 - protein, 18 aa, Artificial
  Eotaxin fragment
GPASVPTTCCFNLANRKI

SEQ ID NO: 15 - protein, 19 aa, Artificial
  Eotaxin fragment
GPASVPTTCCFNLANRKIP

SEQ ID NO: 16 - protein, 20 aa, Artificial
  Eotaxin fragment
GPASVPTTCCFNLANRKIPL

SEQ ID NO: 17 - protein, 21 aa, Artificial
  Eotaxin fragment
GPASVPTTCCFNLANRKIPLQ

SEQ ID NO: 18 - protein, 6 aa, Artificial
  Eotaxin fragment
FNLANR

SEQ ID NO: 19 - protein, 7 aa, Artificial
  Eotaxin fragment
FNLANRK

SEQ ID NO: 20 - protein, 8 aa, Artificial
  Eotaxin fragment
FNLANRKI

SEQ ID NO: 21 - protein, 9 aa, Artificial
  Eotaxin fragment
FNLANRKIP

SEQ ID NO: 22 - protein, 10 aa, Artificial
  Eotaxin fragment
FNLANRKIPL

SEQ ID NO: 23 - protein, 21 aa, Artificial
  Eotaxin fragment
KKKWVQDSMKYLDQKSPTPKP

SEQ ID NO: 24 - protein, 20 aa, Artificial
  Eotaxin fragment
KKWVQDSMKYLDQKSPTPKP

SEQ ID NO: 25 - protein, 19 aa, Artificial
  Eotaxin fragment
KWVQDSMKYLDQKSPTPKP

SEQ ID NO: 26 - protein, 18 aa, Artificial
  Eotaxin fragment
WVQDSMKYLDQKSPTPKP

SEQ ID NO: 27 - protein, 17 aa, Artificial
  Eotaxin fragment
VQDSMKYLDQKSPTPKP

SEQ ID NO: 28 - protein, 16 aa, Artificial
  Eotaxin fragment
QDSMKYLDQKSPTPKP

SEQ ID NO: 29 - protein, 15 aa, Artificial
  Eotaxin fragment
DSMKYLDQKSPTPKP

SEQ ID NO: 30 - protein, 14 aa, Artificial
  Eotaxin fragment
SMKYLDQKSPTPKP

SEQ ID NO: 31 - protein, 13 aa, Artificial
  Eotaxin fragment
MKYLDQKSPTPKP

SEQ ID NO: 32 - protein, 12 aa, Artificial
  Eotaxin fragment
KYLDQKSPTPKP

SEQ ID NO: 33 - protein, 11 aa, Artificial
  Eotaxin fragment
YDLQKSPTPKP

SEQ ID NO: 34 - protein, 10 aa, Artificial
  Eotaxin fragment
LDQKSPTPKP

SEQ ID NO: 35 - protein, 9 aa, Artificial
  Eotaxin fragment
DQKSPTPKP

SEQ ID NO: 36 - protein, 8 aa, Artificial
  Eotaxin fragment
QKSPTPKP

SEQ ID NO: 37 - protein, 7 aa, Artificial
  Eotaxin fragment
KSPTPKP

SEQ ID NO: 38 - protein, 6 aa, Artificial
  Eotaxin fragment
SPTPKP

SEQ ID NO: 39 - protein, 7 aa, Artificial
  Peptide spacer
SSPPPPC

SEQ ID NO: 40 - protein, 6 aa, Artificial
  Peptide spacer
RPPPPC

SEQ ID NO: 41 - protein, 6 aa, Artificial
  Peptide spacer
LPPPPC

SEQ ID NO: 42 - protein, 28 aa, Artificial
  Peptide spacer
SSPPPPCKKKWVQDSMKYLDQKSPTPKP

SEQ ID NO: 43 - protein, 28 aa, Artificial
  Peptide spacer
KKKWVQDSMKYLDQKSPTPKPSSPPPPC

SEQ ID NO: 44 - protein, 28 aa, Artificial
  Peptide spacer
CPPPPSSKKKWVQDSMKYLDQKSPTPKP

SEQ ID NO: 45 - protein, 28 aa, Artificial
  Peptide spacer
KKKWVQDSMKYLDQKSPTPKPCPPPPSS

SEQ ID NO: 46 - protein, 27 aa, Artificial
  Peptide spacer
GPASVPTTCCFNLANRKIPLSSPPPPC

SEQ ID NO: 47 - protein, 27 aa, Artificial
  Peptide spacer
SSPPPPCGPASVPTTCCFNLANRKIPL

SEQ ID NO: 48 - protein, 27 aa, Artificial
  Peptide spacer
CPPPPSSGPASVPTTCCFNLANRKIPL

SEQ ID NO: 49 - protein, 27 aa, Artificial
  Peptide spacer
GPASVPTTCCFNLANRKIPLSSPPPPC

SEQ ID NO: 50 - protein, 17 aa, Artificial
  Eotaxin fragment analog
SGKCPQKAVISSPPPPC

SEQ ID NO: 51 - protein, 17 aa, Artificial
  Eotaxin fragment analog
CPPPPSSSGKCPQKAVI

SEQ ID NO: 52 - protein, 17 aa, Artificial
  Eotaxin fragment analog
FKTKLAKDICSSPPPPC

SEQ ID NO: 53 - protein, 17 aa, Artificial
  Eotaxin fragment analog
CPPPPSSFKTKLAKDIC

SEQ ID NO: 54 - protein, 17 aa, Artificial
  Eotaxin fragment analog
ADPKKKWVQDSSPPPPC

SEQ ID NO: 55 - protein, 17 aa, Artificial
  Eotaxin fragment analog
CPPPPSSADPKKKWVQD

SEQ ID NO: 56 - protein, 17 aa, Artificial
  Eotaxin fragment analog
SGKTPQKAVISSPPPPC

SEQ ID NO: 57 - protein, 17 aa, Artificial
  Eotaxin fragment analog
CPPPPSSSGKTPQKAVI

SEQ ID NO: 58 - protein, 17 aa, Artificial
  Eotaxin fragment analog
FKTKLAKDITSSPPPPC

SEQ ID NO: 59 - protein, 17 aa, Artificial
  Eotaxin fragment analog
CPPPPSSFKTKLAKDIT

SEQ ID NO: 60 - protein, 17 aa, Artificial
  Eotaxin fragment analog
ADPKKKWVQDSSPPPPC

SEQ ID NO: 61 - protein, 17 aa, Artificial
  Eotaxin fragment analog
CPPPPSSADPKKKWVQD

SEQ ID NO: 62 - protein, 7 aa, Artificial
  Antigenic peptide
IPTEIPT

SEQ ID NO: 63 - protein, 8 aa, Artificial
  Antigenic peptide
IPTEIPTS

SEQ ID NO: 64 - protein, 9 aa, Artificial
  Antigenic peptide
IPTEIPTSA

SEQ ID NO: 65 - protein, 10 aa, Artificial
  Antigenic peptide
IPTEIPTSAL

SEQ ID NO: 66 - protein, 11 aa, Artificial
  Antigenic peptide
IPTEIPTSALV

SEQ ID NO: 67 - protein, 12 aa, Artificial
  Antigenic peptide
IPTEIPTSALVK

SEQ ID NO: 68 - protein, 13 aa, Artificial
  Antigenic peptide
IPTEIPTSALVKE

SEQ ID NO: 69 - protein, 14 aa, Artificial
  Antigenic peptide
IPTEIPTSALVKET

SEQ ID NO: 70 - protein, 15 aa, Artificial
  Antigenic peptide
IPTEIPTSALVKETL

SEQ ID NO: 71 - protein, 16 aa, Artificial
  Antigenic peptide
IPTEIPTSALVKETLA

SEQ ID NO: 72 - protein, 17 aa, Artificial
  Antigenic peptide
IPTEIPTSALVKETLAL

SEQ ID NO: 73 - protein, 18 aa, Artificial
  Antigenic peptide
IPTEIPTSALVKETLALL

SEQ ID NO: 74 - protein, 19 aa, Artificial
  Antigenic peptide
IPTEIPTSALVKETLALLS

SEQ ID NO: 75 - protein, 20 aa, Artificial
  Antigenic peptide
IPTEIPTSALVKETLALLST

SEQ ID NO: 76 - protein, 21 aa, Artificial
  Antigenic peptide
IPTEIPTSALVKETLALLSTH

SEQ ID NO: 77 - protein, 22 aa, Artificial
  Antigenic peptide
IPTEIPTSALVKETLALLSTHR

SEQ ID NO: 78 - protein, 23 aa, Artificial
  Antigenic peptide
IPTEIPTSALVKETLALLSTHRT

SEQ ID NO: 79 - protein, 24 aa, Artificial
  Antigenic peptide
IPTEIPTSALVKETLALLSTHRTL

SEQ ID NO: 80 - protein, 25 aa, Artificial
  Antigenic peptide
IPTEIPTSALVKETLALLSTHRTLL

SEQ ID NO: 81 - protein, 26 aa, Artificial
  Antigenic peptide
IPTEIPTSALVKETLALLSTHRTLLI

SEQ ID NO: 82 - protein, 27 aa, Artificial
  Antigenic peptide
IPTEIPTSALVKETLALLSTHRTLLIA

SEQ ID NO: 83 - protein, 28 aa, Artificial
  Antigenic peptide
IPTEIPTSALVKETLALLSTHRTLLIAN

SEQ ID NO: 84 - protein, 29 aa, Artificial
  Antigenic peptide
IPTEIPTSALVKETLALLSTHRTLLIANE

SEQ ID NO: 85 - protein, 30 aa, Artificial
  Antigenic peptide
IPTEIPTSALVKETLALLSTHRTLLIANET

SEQ ID NO: 86 - protein, 31 aa, Artificial
  Antigenic peptide
IPTEIPTSALVKETLALLSTHRTLLIANETL

SEQ ID NO: 87 - protein, 32 aa, Artificial
  Antigenic peptide
IPTEIPTSALVKETLALLSTHRTLLIANETLR

SEQ ID NO: 88 - protein, 33 aa, Artificial
  Antigenic peptide
IPTEIPTSALVKETLALLSTHRTLLIANETLRI

SEQ ID NO: 89 - protein, 34 aa, Artificial
  Antigenic peptide
IPTEIPTSALVKETLALLSTHRTLLIANETLRIP

SEQ ID NO: 90 - protein, 35 aa, Artificial
  Antigenic peptide
IPTEIPTSALVKETLALLSTHRTLLIANETLRIPV

SEQ ID NO: 91 - protein, 19 aa, Artificial
  Antigenic peptide
QLCTEEIFQGIGTLESQTV

SEQ ID NO: 92 - protein, 18 aa, Artificial
  Antigenic peptide
LCTEEIFQGIGTLESQTV

SEQ ID NO: 93 - protein, 17 aa, Artificial
  Antigenic peptide
CTEEIFQGIGTLESQTV

SEQ ID NO: 94 - protein, 16 aa, Artificial
  Antigenic peptide
CTEEIFQGIGTLESQT

SEQ ID NO: 95 - protein, 15 aa, Artificial
  Antigenic peptide
CTEEIFQGIGTLESQ

SEQ ID NO: 96 - protein, 14 aa, Artificial
  Antigenic peptide
CTEEIFQGIGTLES

SEQ ID NO: 97 - protein, 22 aa, Artificial
  Antigenic peptide
TVERLFKNLSLIKKYIDGQKKK

SEQ ID NO: 98 - protein, 21 aa, Artificial
  Antigenic peptide
VERLFKNLSLIKKYIDGQKKK

SEQ ID NO: 99 - protein, 20 aa, Artificial
  Antigenic peptide
VERLFKNLSLIKKYIDGQKK

SEQ ID NO: 100 - protein, 24 aa, Artificial
  Antigenic peptide
RVNQFLDYLQEFLGVMNTEWIIES

SEQ ID NO: 101 - protein, 23 aa, Artificial
  Antigenic peptide
VNQFLDYLQEFLGVMNTEWIIES

SEQ ID NO: 102 - protein, 30 aa, Artificial
  Antigenic peptide
PVHKNHQLCTEEIFQGIGTLESQTVQGGTV

SEQ ID NO: 103 - protein, 30 aa, Artificial
  Antigenic peptide
ERLFKNLSLIKKYIDGQKKKCGEERRRVNQ

SEQ ID NO: 104 - protein, 20 aa, Artificial
  Antigenic peptide
FLDYLQEFLGVMNTEWIIES

SEQ ID NO: 105 - protein, 13 aa, Artificial
  Antigenic peptide
RIPVPVHKNHQLC

SEQ ID NO: 106 - protein, 7 aa, Artificial
  Antigenic peptide
QTVQGGT

SEQ ID NO: 107 - protein, 8 aa, Artificial
  Antigenic peptide
KCGEERRR

SEQ ID NO: 108 - protein, 7 aa, Artificial
  Antigenic peptide
TEEIFQG

SEQ ID NO: 109 - protein, 6 aa, Artificial
  Antigenic peptide
EWIIES

SEQ ID NO: 110 - protein, 9 aa, Artificial
  Antigenic peptide
PVHKNHQLT

SEQ ID NO: 111 - protein, 11 aa, Artificial
  Antigenic peptide
KKYIDGQKKKT

SEQ ID NO: 112 - protein, 8 aa, Artificial
  Antigenic peptide
KKKTGEER

SEQ ID NO: 113 - protein, 10 aa, Artificial
  Antigenic peptide
TGEERRRVNQ

SEQ ID NO: 114 - protein, 15 aa, Artificial
  Antigenic peptide
PVHKNHQLTLPPPPC

SEQ ID NO: 115 - protein, 18 aa, Artificial
  Antigenic peptide
KKYIDGQKKKTSSPPPPC

SEQ ID NO: 116 - protein, 14 aa, Artificial
  Antigenic peptide
CPPPPLKKKTGEER

SEQ ID NO: 117 - protein, 10 aa, Artificial
  Eotaxin fragment
ASVPTTSSFN

SEQ ID NO: 118 - protein, 10 aa, Artificial
  Eotaxin fragment
ANRKIPLQRL

SEQ ID NO: 119 - protein, 10 aa, Artificial
  Eotaxin fragment
ASVPTTCCFN

SEQ ID NO: 120 - protein, 10 aa, Artificial
  Eotaxin fragment analog
ASVPTTAAFN

SEQ ID NO: 121 - protein, 10 aa, Artificial
  Eotaxin fragment analog
ASVPTTSAFN

SEQ ID NO: 122 - protein, 9 aa, Artificial
  Antigenic peptide
KCGEERRRV

SEQ ID NO: 123 - protein, 9 aa, Artificial
  Antigenic peptide
EERRRVNQF

SEQ ID NO: 124 - protein, 10 aa, Artificial
  Eotaxin-2 fragment
VVIPSPSSMF

SEQ ID NO: 125 - protein, 10 aa, Artificial
  Eotaxin-2 fragment
MFFVSKRIPE

SEQ ID NO: 126 - protein, 10 aa, Artificial
  Eotaxin-2 fragment
VSKRIPENRV

SEQ ID NO: 127 - protein, 10 aa, Artificial
  Eotaxin-3 fragment
SDISKTSSFQ

SEQ ID NO: 128 - protein, 10 aa, Artificial
  Eotaxin-3 fragment
FQYSHKPLPW

SEQ ID NO: 129 - protein, 10 aa, Artificial
  Eotaxin-3 fragment
SHKPLPWTWV

SEQ ID NO: 130 - protein, 12 aa, Artificial
  Eotaxin fragment analog
SYRRITSGKSPQ

SEQ ID NO: 131 - protein, 12 aa, Artificial
  Eotaxin fragment analog
SYRRITSGKAPQ

SEQ ID NO: 132 - protein, 12 aa, Artificial
  Eotaxin fragment analog
SYRRITSGKTPQ

SEQ ID NO: 133 - protein, 12 aa, Artificial
  Eotaxin-2 fragment
SYQLSSRSTSLK

SEQ ID NO: 134 - protein, 12 aa, Artificial
  Eotaxin-2 fragment
SYQLSSRSTTLK

SEQ ID NO: 135 - protein, 12 aa, Artificial
  Eotaxin-2 fragment
SYQLSSRSTALK

SEQ ID NO: 136 - protein, 12 aa, Artificial
  Eotaxin-3 fragment
SYEFTSNSSSQE

SEQ ID NO: 137 - protein, 12 aa, Artificial
  Eotaxin-3 fragment
SYEFTSNSTSQE

SEQ ID NO: 138 - protein, 12 aa, Artificial
  Eotaxin-3 fragment
SYEFTSNSASQE

(19) United States
(12) Patent Application Publication
(10) Pub. No.: US 2006/0292115 A1
(43) Pub. Date: Dec. 28, 2006

(54) Methods and compositions for treating and preventing inflammatory conditions

(76) Inventors: Dimitrios T Drivas, Scarsdale, NY (US); Peter Blackburn, New York, NY (US)

Correspondence Address: HOXIE & TSO LLP, 374 MILLBURN AVENUE, SUITE 300 E, MILLBURN, NJ, 07041, US

(21) Appl. No.: 10/550,866

(22) PCT Filed: Mar. 24, 2004
(86) PCT No.: PCT/US04/08901

§ 371 Date: Sep. 23, 2005

Related U.S. Application Data
(60) Provisional application No. 60/457,137, filed on Mar. 24, 2003.

(51) Int. Cl. A61K 39/395; A61K 38/20

(52) U.S. Cl. 424/85.2; 424/131.1

(57) ABSTRACT

Methods of treating eosinophilia by down regulating eotaxin and at least one other Th-2 related cytokine are dislosed as are multivalent immunogenic compositions that generate an active immune response in a subjet comprising autoantibodies to eotaxin-1, eotaxin-2, IL-4, IL-5, IL-9, and IL-13 and treatment methods using such compositions.

### BACKGROUND OF THE INVENTION

Cytokines are peptide messenger molecules that are produced by and act on the cells of the immune system. They are paracrine or autocrine in character and may act systemically if they escape cell binding and spill over to general circulation through the lymph or plasma. While cytokines play a critical role as the chemical messengers of the immune system and are essential to normal immune function, in certain immune system disorders the levels of specific cytokines are abnormal and potentiate the disease state.

In immune system disorders such as atopic conditions, in particular asthma, and in autoimmune diseases, chemokines, a particular class of cytokines, and their subclass interleukins, play an important role. The presence and levels of these chemokines in tissues induce physiological changes, which in individuals suffering from a particular disease are amplified and perpetuated so as to result in a phenotype, which is recognized as the disease state. Chemokines are mediators of the initiation and maintenance of inflammation. Disrupting the chemokine-receptor interactions with neutralizing anti-chemokine antibodies or with chemokine receptor antagonists may diminish or inhibit inflammatory responses. Autoantibodies to chemokines can effectively neutralize chemokines and their signaling and modulatory effects on the immune system and disease. The concept of a therapy for diseases associated with abnormal levels of chemokines through the regulation of a patient's autoantibody levels to the target chemokine may in fact emulate the body's own etiology or regulation of the disease.

The important modulatory role of chemokines in disease has resulted in a number of products in development whose mode of action is to block the binding of the chemokines to their receptors. The majority of these products, some of which are in clinical trials, are based on humanized monoclonal antibodies (“mAbs”), non-antigenic receptor antagonists or soluble receptor molecules or analogues; all of which require many repeat administrations and do not ideally lend themselves to long term therapy or prophylactic treatment. For example humanized anti-TNFalpha mAbs for rheumatoid arthritis and inflammatory bowel disease, and several humanized anti-IL-4, anti-IL-5, anti-IL-8 and anti-IL-9 mAbs for the treatment of asthma are in development. These humanized mAb treatments may have potential for the short term treatment of acute disease states, however, they are not ideally suited for long term maintenance therapy. As a result, therapies which result in an effective harnessing of the patient's own immune system to mount a polyclonal autoantibody based control of the target chemokine levels have been suggested as a means to overcome many of the disadvantages of the products currently in clinical trials (see for example, WO 00/65058 and U.S. Pat. No. 6,093,405).

Cytokine Neutralization

The most prevalent methods of cytokine neutralization under development are by administration of cytokine receptor antagonists, by the administration of humanized monoclonal antibodies against the cytokine or the cytokine receptor, or by the administration of truncated forms of the receptor, which bind to the cytokine and neutralize it. For example, U.S. Pat. Nos. 5,912,136; 5,914,110; 5,959,085; 6,168,791 B1; and 6,171,590 B1 all disclose such methods. Another reported method of neutralization of cytokines is through the use of antisense molecules complementary to the coding sequence of the cytokine gene, the goal of which is to inhibit the expression of the gene.

Cytokine neutralization with autoantibodies generated by active immunization is now considered a promising method of treating pathological conditions (Zagury et al., “Toward a new generation of vaccines: The anti-cytokine therapeutic vaccines”, PNAS, Jul. 3, 2001, Vol. 98, No. 14, 8024-8029, Svenson et al., Journal of Immunological Methods 236 (2000) 1-8, Richard et al., PNAS, Jan. 18, 2000, Vol. 97, No. 2, 767-772. Dalum et al., Nature Biotechnology, Vol. 17, July 1999, 666-669). Vaccines useful for cytokine neutralization can be produced by inactivating the cytokine molecule and rendering it immunogenic, see for example Ciapponi et al., (“Induction of interleukin-6 (IL-6) autoantibodies through vaccination with an engineered 16 receptor antagonist.” Nature Biotechnology, Vol. 15, October 1997, pgs. 997-1001) who successfully demonstrated the neutralization of IL-6 after vaccination with an antigenic, non-biologically active, engineered IL-6 receptor antagonist in transgenic mice with high circulating levels of human IL-6. Ciapponi et al., speculate on the advantage of such a vaccination treatment of immune or neoplastic diseases over therapies with monoclonal antibodies (mAbs) or receptor antagonists, which require continuous parenteral delivery. Alternatively, the cytokine can be coupled to an immunogenic carrier to render it immunogenic (see for example Richard et al., PNAS, Jan. 18, 2000, Vol. 97, No. 2, 767-772, U.S. Pat. No. 6,482,403, U.S. Pat. No. 6,471,957, U.S. Pat. No. 6,455,504, U.S. Pat. No. 6,420,141, WO 01/43771 and WO 00/64397). The anti-cytokine vaccine approach has been proposed for the treatment of asthma and allergic diseases by controlling the levels of interleukins implicated in these disease states, (see WO 00/65058, WO 01/62287 and U.S. Pat. No. 6,093,405).

Atopic Conditions: Asthma, Allergy and Allergic Diseases.

Asthma is becoming one of the most important medical problems with about 15 million asthma sufferers in the US alone. The number of asthma sufferers has increased over 50% in the last 10 years with 700,000 victims, mostly children, emerging in the US each year.

While all humans produce a protective immune response to allergens that enter the lungs, some individuals react by producing an overwhelming response of cells producing the allergic immune antibody, IgE, which release substances, including chemokines, that cause asthma attacks. Chronic asthma, in which asthmatic symptoms are exhibited at least twice a week, is currently treated with two types of drugs: (1) a medication that quells inflammation such as a corticosteroid and (2) a rescue drug to open constricted airways and make breathing easier when attacks occur. The current drugs on the market only help in relieving the symptoms of asthma and do not eliminate or suppress the immune response that causes the allergy and subsequently the asthma. In addition, the majority of current medications are either pills which must be taken frequently or must be administered frequently or during an attack with an inhaler. As predominantly steroid-based therapies they may also result in undesirable side effects and decreased efficacy with increased or long-term use. A vaccine type medication, administered only every few months, that suppresses or eliminates the allergic-like response that results in asthmatic attacks is highly desirable.

T-helper cells perform a key function of the immune response. Generally T-helper precursor (Th-p) cells differentiate as part of the immune response into either T-helper 1 (Th-1) or T-helper 2 (Th-2) effector cells each of which have important biological roles. In response to an antigen, which enters the lungs, an individual can either develop a protective Th-1 or an allergic Th-2 response. The Th-2 response results in the production of IgE antibodies and allergy symptoms. Allergic and asthmatic individuals exhibit an overwhelmingly Th-2 response to inhaled allergens associated with elevated levels of IgE.

The airway inflammation in asthma is characterized by an infiltration of the airway wall by Th2 cells, eosinophils and mast cells. Each of these cells contributes to the physiological changes that characterize asthma and each of the cell types produce and are responsive to a limited panel of cytokines.

The differentiation of Th-p cells into either Th-1 or Th-2 cells is mediated by different hormone signaling pathways comprised mostly of different sets of interleukin chemokines. The Th-2 pathway is mediated by cytokines comprising IL-4, IL-5, IL-6, IL-9, IL-10 and IL-13. These interleukins are produced by Th-2 and other immune system cells and are critical for antibody production and the signaling to other cells involved in the allergic immune response. Eotaxins are another class of chemokines in the Th-2 signaling cascade, which regulate eosinophils. Eotaxins may affect the production of IgE antibodies and play an important role in the allergic response.

There is currently a substantial research and development effort in new asthma therapies. These include the chemokine neutralization therapies primarily for eotaxin, IL-4, IL-5 and IL-13, and other strategies aimed at neutralizing IgE antibodies either by direct blockade with humanized anti-IgE mAbs or possibly a vaccine to immunize against IgE. Other, more conventional allergy vaccine strategies, center on immunization or desensitization with specific peptide allergens, for example, cat dander or ragweed pollen. Research also continues with efforts on new delivery methods and application of DNA vaccine technology to allergen vaccination, however, allergen-specific strategies do not provide a general therapy for asthma.

There is also a considerable research effort for a general asthma vaccine focused on either generating a nonspecific Th-1 immune response, or shifting a patient's Th-2 response to a Th-1 response by immunizing with known Th-1 antigens or DNA vaccines which result in a Th-1 immune response, (see for example U.S. Pat. No. 6,086,898). The active immunization approaches that have been suggested as therapy for allergy and autoimmune disease have focused on controlling levels of the interleukins IL-4 and IL-5. U.S. Pat. No. 6,093,405 discloses inducing an immune response against IL-4 or IL-5 by actively immunizing with an immunogenic IL-4 or IL-5 cytokine composition in order to treat allergy or autoimmune disease respectively. WO 00/65058 discloses the construction of anti-IL-5 immunogens and their use in a method to down-regulate IL-5 in a proposed method of controlling asthma and other chronic allergic diseases.

The current state of the art concerning the development eosinophilia implicated in allergic disorders contemplates primary functions for each of the Th-2 chemokines, which are intricately and integrally related to the functions of the other chemokines in the pathway. For example eotaxin is believed to promote eosinophil accumulation in tissues, while IL-5 increases the induction of eosinophilia in the blood. Eotaxin production in turn may be regulated by IL-13 and IL-13 can regulate eosinophil migration independent of IL-5. It has been reported that in mice a deficiency in the production of IL-5, eotaxin or both predisposes mice to an intrinsic defect in T-cells which subsequently impairs the ability of CD4+ T-cells to produce IL-13 (see Mattes, et al., J. Exp. Med., Vol. 195, No. 11, Jun. 3, 2002, 1433-1444).

Eotaxins

Eotaxins are eosinophil-specific chemokines, which stimulate eosinophil accumulation or attract eosinophils. Eotaxins induce chemotaxis of eosinophils but do not significantly induce the chemotaxis of neutrophils, monocytes or T-cells. The eotaxins are members of the CC subfamily of chemokines, a class which also includes monocyte chemotactic proteins (MCPs) and macrophage inflammatory proteins (MIPs), see: Van Coillie et al., Cytokine & Growth Factor Reviews, 10 (1999) 61-86; Garcia-Zepeda, et al. (1996) Nat. Med., 2: 449-456.

There are currently at least three molecules classified as eotaxins; the first to be identified, eotaxin-1 and still referred to as eotaxin, (see Kitaura, M. et al., J. Biol. Chem., 1996, 271; 7725-30 and Ponath et al., J. Clin. Invest. 1996, 97: 604-12.) and the later discovered eotaxin-2 and eotaxin-3, (see Conroy et al. Respir Res 2001, 2: 150-156; Guiterrez-Ramos et al. Immunology Today, November 1999, Vol. 20, No. 11, 500-504). Eotaxin binds to and acts through the chemokine receptor 3, CCR3, with relatively high affinity to induce eosinophil recruitment. The structure and peptide sequence and the genes which encode eotaxins are known, and receptor binding has been studied and characterized (see Garcia-Zepeda et al. Nature Medicine, Vol. 2, No. 4, April 1996, 449456; Ye et al., The Journal of Biological Chemistry, Vol. 275, No. 35, Sep. 1, 2000 27250-27257; Mayer and Stone, The Journal of Biological Chemistry, Vol. 276, No. 17, Apr. 27, 2001, 13911-13916.

Eosinophils are one of the principle components of the body's Th-2-type immune defense to helmitic parasitic infections and accumulate in the blood and tissues of infected individuals. The eosinophils contain granules of cationic proteins, which upon degranulation are released into the cell's environment and damage the invading helminth. Atopic conditions such as asthma and chronic allergic diseases are characterized by a predominant Th-2 type immune response to allergic non-helmitic stimuli. Inflammation of the lung in patients with asthma and chronic allergic diseases is characterized by infiltration and accumulation in the lung and in particular of the bronchial mucosa of eosinophils. In these conditions in the absence of helmitic infection the release of the eosinophil's cationic proteins upon degranulation damages the surrounding cells. As a result, eotaxin has been recognized as a potential target for the treatment and prevention of atopic conditions and in particular the therapy of asthma and allergic disease. U.S. Pat. Nos. 5,993,814 and 6,031,080 and PCT publications WO 95/07985, WO 97/00960, WO 97/12914, and WO 99/10534 suggest the use in therapy of various eotaxin agonists and antagonists including antibodies against eotaxin. WO 01/66754 discloses the production and use of anti-eotaxin human antibodies CAT 212 and 213 and fragments thereof for the treatment of eotaxin mediated conditions in a passive immunization regimen.

The receptor on which eotaxin acts, the CC, CKR3 or CXCR3 receptor, has been characterized, (see WO 97/41154 and U.S. Pat. No. 6,171,590 B1) and agonists and antagonists of this receptor have also been suggested for therapy (see U.S. Pat. No. 6,271,347). U.S. Pat. No. 6,171,590 B1 suggests that immunogenic oligopeptides derived from the receptor can be used in active immunization against the receptor for a therapeutic effect. None of the publications or patents referred to above, however, suggest the active immunization against eotaxin itself as a therapeutic method or disclose immunogenic compositions useful for such active immunotherapy.

The current invention provides compositions that are useful for the treatment of conditions characterized by eosinophil accumulation. These atopic conditions of which asthma and chronic allergic diseases are the most prevalent include atopic skin conditions such as psoriasis and other conditions such as eosinophilic ulcerative colitis. In each of these conditions eosinophils accumulate in the affected tissue to a large extent through eotaxin induced eosinophil recruitment. The chronic presence of elevated levels of eosinophils in the affected tissues results in significant tissue damage which over time progresses and may become irreversible. The cytokine neutralization therapies that are being pursued by others for the most part are directed to blocking the action of only one chemokine such as IL-4, IL-5, IL-9, IL-13 or eotaxin or of the chemokine on its receptor such as the chemokine, eotaxin on the CCR3 receptor. These therapies utilize small molecule antagonists, passive immunization with human or humanized monoclonal antibodies, active immunization against one cytokine or passive or active immunization against the receptor itself. The small molecule and passive immunization approaches require repeat administration and suffer from the standpoint of patient compliance. Furthermore, the induction of neutralizing antibodies to the administered mAbs as a result of repeat therapy can seriously compromise the effectiveness of passive immunotherapy with mAbs for long term treatment of a chronic disease (Adair, F., Drug Discovery World, Summer 2002 pp 53-59). On the other hand active immunization against the receptor itself may interfere with the binding of other chemokines to the receptor and may have unforeseen and unintended biological consequences.

### SUMMARY OF THE INVENTION

The invention provides methods and compositions for the treatment of cytokine-mediated disorders that involve immunomodulatory pathways of more than one cytokine. Such disorders include autoimmune disorders and atopic conditions such as asthma and allergic conditions which involve the Th1 and Th2 cytokine pathways. The method of the invention provides for the blockage of the activity of two or more cytokines whose functions are related in the pathway. The overall effect of the cytokine blockade is to down regulate the overall levels of cytokines and the associated immune response and thus ameliorate the condition being treated.

The methods of the invention contemplate the immunization of a subject so as to generate an immune response in a subject of autoantibodies against two or more cytokines in the cytokine pathway of interest. The immunogenic compositions of the invention may be used to generate an effective immune response in the immunized subject specific for the target cytokines. The methods may comprise separate administration of immunogens each of which is directed to generating an immune response against only one cytokine target or the administration of a multivalent immunogen capable of generating an immune response to two or more cytokine targets.

According to the invention the Th-2 immune response related disorders may be treated or prevented by regulating Th-2 chemokine levels and blockading the effect in the subject of the chemokines. This may be achieved in one method by actively immunizing the subject against one or more of the Th-2 chemokines which may have an effect on the disorder. For example the subject may be actively immunized against eotaxin, while at the same time blockading at least one other cytokine in the Th-2 pathway such as IL-4, IL-5, IL-9 or IL-13. The concurrent blockade can be achieved by any method including: active immunization against the target cytokines; passive immunization using antibodies; the administration of antagonists which prevent the binding of the target cytokines to their receptors; the administration of cytokine traps, agents such as receptor fragments which bind to freely circulating cytokines and neutralize their activity; or by a combination of any of these methods or compositions. For example the treatment of eosinophilia may be achieved by concurrently blocking: eotaxin and IL-5; eotaxin and IL-13; eotaxin, IL-5, and IL-13, eotaxin and IL-9, or eotaxin and IL-4. In other embodiments of the invention in addition to the blockade of eotaxin, eotaxin 2 and/or eotaxin 3 may also be blocked. The various target cytokines may be effectively blocked by active immunization of animal subjects, including humans, with anti-cytokine immunogens.

In a particular embodiment of the invention the blockade of eotaxin is achieved by the active immunization against eotaxin. The blockade of the additional cytokine or cytokines may be achieved by any means including active immunization. The active immunization may be achieved through the administration of separate therapeutic vaccine products each directed at one of the target cytokines or alternatively through the administration of one multivalent therapeutic vaccine product capable of inducing an active immune response to all of the target cytokines. The active immunization can be achieved by any method of vaccination including antigenic peptide immunogenic protein conjugates or DNA vaccines.

Specific embodiments of the invention concern multivalent vaccine products targeting more than one cytokine involved in the Th-2 immune response. For example, in one embodiment the invention provides a method of treating allergic disorders and asthma by actively immunizing a patient against two of the cytokines involved in the Th-2 pathway, IL-5 and eotaxin. In a related embodiment the invention provides a multivalent immunogen capable of eliciting autoantibodies in a subject to the cytokines IL-5 and eotaxin and the use of such multivalent immunogens to treat inflammatory conditions which involve eosinophil accumulation such as asthma and allergic diseases and other atopic conditions. The multivalent vaccine or immunogenic products may employ different forms of immunogen and delivery methods known in the art and can be used alone or in combination with other therapeutic agents.

The immunogenic compositions may be used to generate an autoantibody response in the patient to IL-5 and eotaxin at levels which are sufficient to immunomodulate or immunoneutralize and thus down regulate the activity of these cytokines and result in a reduction of eosinophil accumulation so as to ameliorate the inflammatory condition. The immunogens may include: combination peptide immunogens comprising portions of the eotaxin and IL-5 sequences or mimetics coupled to an immunogenic carrier or DNA vaccines encoding such combination peptide immunogens.

The invention also concerns methods for the treatment of conditions associated with eosinophilia or eosinophil accumulation comprising the active immunization of a subject against eotaxin and IL-5 with the immunogenic compositions of the invention. The inventive treatment methods include therapies that involve the treatment of the subject with other pharmaceutical agents in addition to the active immunization using the multivalent anti-eotaxin and anti-IL-5 vaccine.

Certain embodiments of the invention concern vaccine compositions which down regulate the Th-2 cytokines in animal subjects, including humans. These vaccine compositions are used to generate an active immune response in the subject comprising autoantibodies to two or more Th-2 chemokines such as eotaxin-1, eotaxin-2, eotaxin-3, IL-5, IL-9, IL-13 and IL-4. The multivalent vaccine products may be used in the treatment of inflammatory conditions that result from eotaxin mediated eosinophil accumulation such as asthma and allergic diseases and other atopic conditions. The vaccine or immunogenic products employ different immunogen types and delivery methods and can be used alone or in combination with other therapeutic agents. In addition the multivalent immunogens may comprise multiple different peptide epitopes for each of the different target chemokines.

The antigenic peptide-based products may be formulated using a modified chemokine that is rendered inactive and immunogenic. In certain embodiments the immunogens comprise at least one chemokine receptor antagonist or agonist, or a chemokine-derived epitope conjugated to an immunogenic carrier. Immunogens derived from the target chemokines or chemokine mimetics can be constructed using methods well known in the art and used to elicit an immune response in laboratory animals such as mice or rabbits. The resulting antibodies can be screened for their ability to neutralize binding of the target chemokine to its receptor in vitro and/or in vivo as a prelude to selecting the most appropriate epitope for clinical development. The DNA based products comprise DNA vaccine products that encode and result in the production in the treated subject of the antigenic peptide products, which will elicit an immune response against the target chemokines in the immunized subject. The design of any particular product depends on the target tissue in which the primary immune response is sought and the type of immune response which will be primarily generated.

In order to construct the immunogens of the invention a specific peptide epitope sequence is coupled to an immunogenic carrier. The resulting immunogen when administered to an animal subject will elicit a humoral immune response which will produce autoantibodies in the subject, which can bind to and neutralize the biological effect of the target chemokine, e.g. IL-5 or eotaxin, The immune response may be maintained for a sustained period by booster administrations of the immunogen. Suitable immunogenic carriers may include proteins or protein toxoids such as Diphtheria toxoid (DT) or Tetanus toxoid (TT), Keyhole limpet hemocyanin (KLH), Influenza virus haemaglutinin, etc. A specific peptide sequence is coupled to the immunogenic carrier via conjugation with bifunctional cross-linking agents. Alternatively, the specific peptide may be coupled to a colloidal metal particle to render it immunogenic or it may be synthesized in tandem with a suitable T-cell epitope sequence(s) as a synthetic heterofunctional immunogenic peptide carrying the specific B-cell epitope and T-cell epitopes to elicit a sustained immunogenic response to the desired chemokine, e.g. eotaxin and IL-5, target fragments.

The invention also concerns methods for the treatment of conditions associated with eotaxin mediated eosinophil accumulation comprising the active immunization of a subject against Th-2 chemokines with the immunogenic compositions of the invention. The inventive treatment methods include therapies, which involve the treatment of the subject with other pharmaceutical agents in addition to the active immunization using the anti-chemokine vaccines.

### DETAILED DESCRIPTION OF THE INVENTION

The invention provides methods and compositions for treating cytokine-mediated disorders in animal subjects including mammals and humans. A particular focus of the invention is the treatment of disorders that involve immunomodulatory pathways made up of multiple cytokines such as the Th-1 and Th-2, T-cell pathways. According to the invention the subject in need of therapy is actively immunized against two or more of the cytokines in the pathway implicated in the disorder being treated. The subject may be immunized with a plurality of separate univalent compositions each directed against a single cytokine or alternatively with a multivalent immunogenic composition capable of eliciting an immune response in a subject comprising autoantibodies against two or more cytokines.

A specific embodiment of the invention provides methods and compositions for the treatment of disorders that involve the Th-2 immunomodulatory pathway and in particular allergic disorders and asthma. Some of these disorders are characterized by eosinophil recruitment to particular tissues such as those of the lung and resulting tissue inflammation. The methods of the invention provide for the active immunization of a subject against multiple cytokines involved in the Th-2 immune response and in a particular embodiment against two of the cytokines involved in the Th-2 immune response, IL-5 and eotaxin, in order to control eosinophil production and recruitment, and to result in a reduction in tissue inflammation.

The present invention provides immunogenic compositions which are useful for active immunization and which are designed to induce a sustained immune response against multiple cytokines involved in a immunomodulatory pathway implicated in a disorder. In one embodiment, the invention provides an immunogen designed to induce a specific immune response against eotaxin and IL-5 which are involved in the Th-2 pathway and eosinophil accumulation implicated in allergic disorders and asthma in animals and humans. Various immunogens according to the invention may be produced and analyzed in the appropriate animal model for the disease or inflammatory condition of interest in order to select the specific immunogen that is optimal for the treatment of the particular condition. Suitable animal models for asthma and other allergic disorders are well known in the art (see Humbles et al., J. Exp. Med., Vol. 186, No. 4, Aug. 18, 1997, 601-612, Corry et al., J. Exp. Med., Vol. 183, January 1996, 109-117, Foster et al., J. Exp. Med., Vol. 183, January 1996, 195-201, Lukacs, et al., Am. J. Respir. Cell Mol. Bio., Vol. 10, 526-532, 1994).

The inventive immunogens should induce an immune response in the subject comprising antibodies of a sufficiently high specificity and binding affinity to the target cytokines so as to be able to neutralize or modulate the biological activity of the target cytokines. In the therapeutic compositions used to treat disorders related to elevated levels of eosinophils the titer of anti-cytokine antibodies induced should be sufficient to result in the lowering of elevated levels of eotaxin in the subject and a reduction in the recruitment of eosinophils to the tissues which are affected by the atopic condition. By actively immunizing the subject or patient with the immunogenic compositions of the invention, an overall level of different autoantibodies, which react with eotaxin and at least one other cytokine which affects eotaxin levels or the Th-2 mediated allergic response such as IL-5, IL-13 or IL-4, are maintained in the subject, to prevent or ameliorate eosinophil recruitment during an allergic reaction. This anti-cytokine autoantibody level can be maintained with booster administration of the inventive immunogenic compositions and thus should provide superior protection for management of the chronic disease state compared to that afforded by small molecule chemokine antagonists or passive anti-chemokine immunization which will exhibit considerable fluctuation in levels of the therapeutic agent, and suffer from less favorable patient compliance. In addition passive immunization of mAbs is subject to development of neutralizing antibodies to the targeted-mAbs upon repeat dosing limiting their effectiveness for long term treatment.

The normally non-immunogenic chemokine such as eotaxin or fragments thereof can also be rendered immunogenic and otherwise biologically inactive by coupling the peptide or fragment to an immunogenic carrier protein or protein toxoid such as Diptheria Toxoid (DT), Tetanus Toxoid (TT), Keyhole Limpet Hemocyanin (KLH), BCG, OVA or others, by well known methods (see for example: U.S. Pat. Nos. 6,217,881; 6,132,720; 5,891,992; 5,609,870; 5,607,676; 5,468,494; 5,023,077; and 4,201,770 and Richard et al., PNAS, Jan. 18, 2000, Vol. 97, No. 2, 767-772; Svenson et al., Journal of Immunological Methods, 236 (2000), 1-8; Dalum et al., Nature Biotechnology, Vol. 17, July 1999, 666-669; Gonzalez et al., Annals of Oncology, 9: 431-435, 1998; and Dalum et al., The Journal of Immunology, 1996, 157: 4796-4804). Alternatively, the chemokines can be conjugated to colloidal metals to render them immunogenic, see U.S. Pat. Nos. 5,112,606, 6,274,552, and 6,528,051, or modified chemokine variants or forms which are inactive but immunogenic may be produced by introducing T helper epitopes in tandem to the eotaxin sequence by using the methods disclosed in WO 00/65058 and WO 95/05849.

A number of chemical, physical and immunological treatments are known which may be useful in producing inactive but immunogenic form of the target chemokine or fragments thereof, (see for example: U.S. Pat. No. 6,093,405; Zagury et al., PNAS, Jul. 3, 2001, Vol. 98, No. 14, 8024-8029; Gringeri et al., Journal of Acquired Immune Deficiency Syndrome and Human Retrovirology, Vol. 20, No. 4, Apr. 1, 1999; Ciapponi et al., Nature Biotechnology, October 1997, Vol. 15, 997-1001; Raaberg et al., Pediatric Research, Vol. 37, No.2, 1995, 169-174; Raaberg et al., Pediatric Research, Vol. 37, No. 2, 1995, 175-181; Gringeri et al., Journal of Acquired Immune Deficiency Syndromes, Vol. 7, No. 7, 1994, 978-988; Zagury et al., Journal of Acquired Immune Deficiency Syndromes, Vol. 5, No. 7, 1992, 676-681).

One embodiment of the invention concerns conjugate immunogens which comprise a plurality of different peptide fragments from the same target chemokine corresponding to desired epitopes on the chemokine molecule which are conjugated to an immunogenic protein carrier such as DT or TT thereby providing promiscuous T-cell epitopes and enabling the immune memory for prolonged antibody response. Such immunogens may be administered to human or animal subjects to develop an active humoral immune response to the chemokine. One embodiment of the invention comprises the entire chemokine such as the human eotaxin molecule conjugated to an immunogenic carrier protein such as DT to render it immunogenic. Other embodiments of the invention comprise various shorter chemokine peptide fragments conjugated to an immunogenic carrier protein.

The conjugates may be constructed using peptides of approximately between 4 and 50 amino acid residues that comprise an epitope or epitopes, which will induce antibodies in the subject which will cross-react with epitopes present on the chemokine molecules existing in the subject. The peptide or peptides comprising the epitopes are then conjugated to the protein carrier in a range of peptide to carrier protein molar ratios. The peptide may be conjugated directly to the immunogenic protein or may incorporate a peptide spacer sequence to extend the desired epitope from the carrier molecule in order to enhance its presentation to antigen presenting cells and thereby the immunogenicity of the desired epitope. The peptide spacer can be attached to either end, the amino or carboxy terminal, of the peptide fragment and the spacer is in turn conjugated to the immunogenic carrier. The conjugation of the peptide to the carrier is accomplished using cross-linking agents, either homobifunctional or heterobifunctional, to attach the desired epitope-containing peptide to the carrier protein. The choice of bifunctional cross-linking agent will depend upon the availability of functional moieties on the peptide. The chemistry for these coupling methods is well known in the art and is set forth in the disclosure of U.S. Pat. Nos. 6,132,720; 5,609,870; and 5,468,494, and Chemistry of Protein Conjugation and Cross-linking, S. S. Wong (1991) CRC Press, Inc.

Alternatively, immunogenic chemokine peptides may be constructed by synthetic peptide chemistry so as to produce in tandem the selected chemokine epitope fragment with a T-cell epitope or epitopes, thereby presenting the selected B-cell epitope (derived from the chemokine) with a promiscuous T-cell epitope to provide for a sustained immune response in the immunized subject.

The conjugate immunogens of the invention may be formulated with adjuvants or other immunostimulatory agents in a pharmaceutically acceptable vehicle with components and using methods well known in the art, (see: Vaccine Design, The Subunit and Adjuvant Approach, (1995) Powell and Newman Eds., Plenum Press (New York), Aucouturier et al., Vaccine 19 (2001) 2666-2672).

Immunogenic chemokine peptides may also be constructed by recombinant DNA technology to produce a plasmid vector in which the desired DNA sequence(s) for the chemokine fragment or multiple fragments from the same or different chemokines is encoded in tandem with the requisite DNA sequence for T-cell epitope(s), thereby producing a fusion protein comprising the selected B-cell epitope (derived from the chemokine) with a T-cell epitope(s). The fusion protein can be expressed in vitro using cell culture/fermentation techniques and can be purified from the culture, or can be exhibited on the plasmid surface, or the plasmid DNA construct can be used as a DNA vaccine. Any of these methods can provide a preparation suitable for eliciting a chemokine-specific and sustained immune response in the subject immunized with the respective preparation.

### EXAMPLE 1

### Anti-Eotaxin Immunogens

Mature human eotaxin-1 is derived from a 97 amino acid precursor protein which contains a 23 amino acid hydrophilic amino terminal sequence which is cleaved off to leave the mature protein of 74 amino acids and approximate molecular weight of 8.4 kDa (see, U.S. Pat. No. 6,403,782, WO 99/10534; WO 97/00960; Ye et al., Journal of Biological Chemistry, Vol. 275, No. 35, Sep. 1, 2000, 27250-27257; Garcia-Zepeda et al., Nature Medicine, Vol. 2, N0. 4, April 1996, 449-45; Ponath et al., J. Clin. Invest., Vol. 97, No. 3, February 1996, 604-612; Mayer et al., Journal Biological Chemistry, Vol. 278, No. 17, Apr. 27, 2001, 13911-13916). The amino acid sequence of mature human eotaxin (SEQ ID NO 1) is as follows (using the one letter code for each amino acid residue):

GPASVPTTCC¹⁰ FNLANRKIPL²⁰ QRLESYRRIT³⁰
SGKCPQKAVI⁴⁰ FKTKLAKDIC⁵⁰ ADPKKKWVQD⁶⁰
SMKYLDQKSP⁷⁰ TPKP⁷⁴

Eotaxin is not normally immunogenic. The peptide itself or fragments of the peptide corresponding to epitopes of interest can be rendered immunogenic by methods well known in the art. One method that may be used is to produce inactive eotaxins or inactive eotaxin fragments which have lost eotaxin biological activity but which are in an immunogenic form and can elicit anti-eotaxin neutralizing antibodies in an animal or human subject. A number of chemical, physical and immunological treatments are known which may be useful in producing inactive but immunogenic eotaxin or fragments thereof, (see for example: U.S. Pat. No. 6,093,405; Zagury et al., PNAS, Jul. 3, 2001, Vol. 98, No. 14, 8024-8029; Gringeri et al., Journal of Acquired Immune Deficiency Syndrome and Human Retrovirology, Vol. 20, No. 4, Apr. 1, 1999; Ciapponi et al., Nature Biotechnology, October 1997, Vol. 15, 997-1001; Raaberg et al., Pediatric Research, Vol. 37, No. 2, 1995, 169-174; Raaberg et al., Pediatric Research, Vol. 37, No. 2, 1995, 175-181; Gringeri et al., Journal of Acquired Immune Deficiency Syndromes, Vol. 7, No. 7, 1994, 978-988; Zagury et al., Journal of Acquired Immune Deficiency Syndromes, Vol. 5, No. 7, 1992, 676-681).

Referring to the mature human eotaxin sequence, in certain embodiments of the invention peptide fragments from amino acid residue 1-45 from the amino terminal end of the molecule and fragments from residue 54 to 74, which constitute the carboxyl terminal end are useful in constructing the immunogen conjugates of the invention. The immunogenic conjugate may comprise one or more different eotaxin epitopes that may be present on the same peptide fragment or on different peptide fragments conjugated to the same immunogenic carrier. Some eotaxin peptide fragments useful in the construction of the immunogens of the invention are as follows:

| | |
|---|---|
| GPASVP; | (SEQ ID NO 2) |
| GPASVPT; | (SEQ ID NO 3) |
| GPASVPTT; | (SEQ ID NO 4) |
| GPASVPTTC; | (SEQ ID NO 5) |
| GPASVPTTCC; | (SEQ ID NO 6) |
| GPASVPTTCCF; | (SEQ ID NO 7) |
| GPASVPTTCCFN; | (SEQ ID NO 8) |
| GPASVPTTCCFNL; | (SEQ ID NO 9) |
| GPASVPTTCCFNLA; | (SEQ ID NO 10) |
| GPASVPTTCCFNLAN; | (SEQ ID NO 11) |
| GPASVPTTCCFNLANR; | (SEQ ID NO 12) |
| GPASVPTTCCFNLANRK; | (SEQ ID NO 13) |
| GPASVPTTCCFNLANRKI; | (SEQ ID NO 14) |
| GPASVPTTCCFNLANRKIP; | (SEQ ID NO 15) |
| GPASVPTTCCFNLANRKIPL; | (SEQ ID NO 16) |
| GPASVPTTCCFNLANRKIPLQ; | (SEQ ID NO 17) |
| FNLANR; | (SEQ ID NO 18) |
| FNLANRK; | (SEQ ID NO 19) |
| FNLANRKI; | (SEQ ID NO 20) |
| FNLANRKIP; | (SEQ ID NO 21) |
| FNLANRKIPL; | (SEQ ID NO 22) |
| KKKWVQDSMKYLDQKSPTPKP; | (SEQ ID NO 23) |
| KKWVQDSMKYLDQKSPTPKP; | (SEQ ID NO 24) |
| KWVQDSMKYLDQKSPTPKP; | (SEQ ID NO 25) |
| WVQDSMKYLDQKSPTPKP; | (SEQ ID NO 26) |
| VQDSMKYLDQKSPTPKP; | (SEQ ID NO 27) |
| QDSMKYLDQKSPTPKP; | (SEQ ID NO 28) |
| DSMKYLDQKSPTPKP; | (SEQ ID NO 29) |
| SMKYLDQKSPTPKP; | (SEQ ID NO 30) |
| MKYLDQKSPTPKP; | (SEQ ID NO 31) |
| KYLDQKSPTPKP; | (SEQ ID NO 32) |
| YLDQKSPTPKP; | (SEQ ID NO 33) |
| LDQKSPTPKP; | (SEQ ID NO 34) |
| DQKSPTPKP; | (SEQ ID NO 35) |
| QKSPTPKP; | (SEQ ID NO 36) |
| KSPTPKTP; | (SEQ ID NO 37) |
| SPTPKP; | (SEQ ID NO 38) |
| ASVPTTSSFN; | (SEQ ID NO 117) |
| ANRKIPLQRL; | (SEQ ID NO 118) |
| and | |
| ASVPTTCCFN. | (SEQ ID NO 119) |

The peptides may be produced by synthetic or recombinant means, which are well known in the art. One skilled in the art will also understand that the amino acid sequence of any peptide fragment may be modified so as to increase its immunogenicity or in order to impart or enhance some other property of the fragment, such as for example charge, charge density, hydrophobicity, to alter solubility or to reduce the potential for oligomerization and aggregation, including the substitution of cysteine resdues to eliminate oligomerization via disulfides, while maintaining its ability to contribute to the induction of an immune response to eotaxin in the subject. Such modifications could be made for example by derivatizing an amino acid residue or by substitution of a particular amino acid for another or by some other method known in the art.

Peptidomimetics or immunomimics, which do not exhibit eotaxin biological activity in the particular animal subject, may also be used to construct the conjugate immunogens. The peptidomimetics may not in and of themselves be immunogenic but may be rendered immunogenic by coupling to an immunogenic peptide. In certain embodiments the peptidomimetics may be derived from other mammalian eotaxin molecules such as mouse or guinea pig eotaxin (see U.S. Pat. Nos. 6,031,080 and 5,993,814).

The eotaxin peptide fragments or immunomimics may be conjugated directly to the immunogenic protein carrier or alternatively may incorporate a peptide spacer sequence to extend the desired epitope from the carrier molecule in order to enhance its presentation to antigen presenting cells and thereby the immunogenicity of the desired epitope. A variety of peptide spacers may be used. U.S. Pat. Nos. 5,609,870 and 5,468,494 disclose peptide spacers and methods of conjugating the spacers to peptides of interest and in turn to immunogenic protein carriers such as DT or TT which may be useful in constructing the conjugate immunogens of the invention. The peptide spacers; SSPPPPC (SEQ ID NO 39), RPPPPC (SEQ ID NO 40) and LPPPPC (SEQ ID NO 41) may be used for the eotaxin peptide fragments of the invention. The spacer peptides may be incorporated at either the amino terminal or carboxyl terminal end of the eotaxin peptide fragment to produce the peptides which are coupled to the immunogenic protein such as for example:

| | |
|---|---|
| SSPPPPCKKKWVQDSMKYLDQKSPTPKP, | (SEQ ID NO 42) |
| KKKWVQDSMKYLDQKSPTPKPSSPPPPC, | (SEQ ID NO 43) |
| CPPPPSSKKKWVQDSMKYLDQKSPTPKP, | (SEQ ID NO 44) |
| KKKWVQDSMKYLDQKSPTPKPGPPPPSS, | (SEQ ID NO 45) |
| GPASVPTTCCFNLANRKIPLSSPPPPC, | (SEQ ID NO 46) |
| SSPPPPCGPASVPTTCCFNLANRKIPL, | (SEQ ID NO 47) |
| CPPPPSSGPASVPTTCCFNLANRKIPL, | (SEQ ID NO 48) |
| and | |
| GPASVPTTCCFNLANRKIPLSSPPPPC. | (SEQ ID NO 49) |

Typically, the spacer sequences are incorporated into specific eotaxin sequences by synthetic peptide chemistry during preparation of the epitope-containing peptide fragments. In particular, eotaxin fragments which contain sequences with number hydrophilic sequences and which are more likely presented at the surface of the molecule are particularly useful for the invention. These include for example:

| | |
|---|---|
| SGKCPQKAVISSPPPPC, | (SEQ ID NO 50) |
| CPPPPSSSGKCPQKAVI, | (SEQ ID NO 51) |
| FKTKLAKDICSSPPPPC, | (SEQ ID NO 52) |
| CPPPPSSFKTKLAKDIC, | (SEQ ID NO 53) |
| ADPKKKWVQDSSPPPPC, | (SEQ ID NO 54) |
| and | |
| CPPPPSSADPKKKWVQD. | (SEQ ID NO 55) |

In one embodiment, to facilitate the conjugation of the specific eotaxin fragments to carrier proteins such as DT and TT using cross-linking agents it is desirable to eliminate cysteine residues, from the natural sequence by substitution with threonine, serine or alanine residues. This substitution of amino acids to eliminate cysteine in the peptide fragments applies to any of the immunogens of the invention and for any target chemokine. Thereby, the hydrodynamic quality of the peptide fragment is retained whilst eliminating potentially detrimental side reactions during the cross-linking step. Examples include:

| | |
|---|---|
| SGKTPQKAVISSPPPPC, | (SEQ ID NO 56) |
| CPPPPSSSGKTPQKAVI, | (SEQ ID NO 57) |
| FKTKLAKDITSSPPPPC, | (SEQ ID NO 58) |
| CPPPPSSFKTKLAKDIT, | (SEQ ID NO 59) |
| ADPKKKWVQDSSPPPPC, | (SEQ ID NO 60) |
| CPPPPSSADPKKKWVQD, | (SEQ ID NO 61) |
| ASVPTTAAFN, | (SEQ ID NO 120) |
| ASVPTTSAFN, | (SEQ ID NO 121) |
| SYRRITSGKSPQ, | (SEQ ID NO 130) |
| SYRRITSGKAPQ | (SEQ ID NO 131) |
| and | |
| SYRRITSGKTPQ. | (SEQ ID NO 132) |

The anti-eotaxin immunogens may comprise one peptide fragment conjugated to the immunogenic carrier, for example one or more copies of a peptide fragment of the sequence GPASVPTTCCFNLANRKIPL (SEQ ID NO 16) conjugated to DT. In other embodiments, two or more different peptide fragments may be conjugated to the same immunogenic carrier so as to induce an active immune response in the subject with antibodies directed to two or more epitopes on eotaxin. Such an immunogen may for example comprise multiple copies of each of the peptide sequences GPASVPTTCCFNLANRKIPL (SEQ ID NO 16) and KKKWVQDSMKYLDQKSPTTKP (SEQ ID NO 23) conjugated to DT.

In another embodiment a formulation may be prepared using two or more different peptide immunogenic carrier conjugates, so as to induce an active immune response in the subject with antibodies directed to two or more epitopes on eotaxin. For example the composition administered will be a co-formulated mixture of two different immunogen constructs, the first comprising one specific epitope conjugated to the carrier with the second construct comprising a second distinct epitope conjugated to other molecules of the carrier. Such an immunogen formulation may for example comprise multiple copies of each of the peptide sequence DT conjugates GPASVPTTCCFNLANRKIPL (SEQ ID NO 16) conjugated to DT; and KKKWVQDSMKYLDQKSPTPKP (SEQ ID NO 23) conjugated to DT.

The eotaxin-toxoid conjugates can be prepared as single entities suitable for formulation individually or for commingling to provide two or more epitope-specific conjugates in a final formulation. Alternatively, the coupling of two or more eotaxin-specific peptides can be coupled via their terminal sulfhydryl groups to a single maleimidyl-toxoid preparation. This is accomplished by reacting the maleimidyl-toxoid with a 1:1 mixture of the eotaxin-specific peptides at a combined 1.1 mole excess of the combined peptides over the available maleimidyl moieties of the activated-toxoid. Thereby, a single toxoid conjugate carrying multiple eotaxin-specific epitopes is accomplished.

In certain embodiments an immune response to multiple epitopes on eotaxin may be induced in the immunized subject to result in a potentially synergistic binding and neutralization of the target eotaxin. Appropriate epitope sequences are selected from those sequences sufficiently removed from one another on the target molecule as to reduce the likelihood for interference between the binding of antibodies to their specific eotaxin epitopes. One embodiment of such a combination of eotaxin epitopes includes the following eotaxin and eotaxin analogue sequences:

| | |
|---|---|
| SGKTPQKAVISSPPPPC | (SEQ ID NO 56) |
| or | |
| CPPPPSSSGKTPQKAVI | (SEQ ID NO 57) |
| in combination with | |
| ADPKKKWVQDSSPPPPC | (SEQ ID NO 60) |
| or | |
| CPPPPSSADPKKKWVQD. | (SEQ ID NO 61) |

### EXAMPLE 2

### Anti-IL-5 Immunogens

Immunogens for raising an active immune response against IL-5 in mammals and humans are known in the art. WO 00/65058 discloses anti IL-5 immunogenic compositions and methods for making and administering them. The anti-IL-5 immunogens disclosed in WO 00/65058 are useful in the methods of the present invention and the disclosure of WO 00/65058 is hereby incorporated by reference in its entirety.

IL-5 peptide fragments useful for constructing the immunogens of the invention are:

| | |
|---|---|
| KCGEERRRV | (SEQ ID NO 122) |
| and | |
| EERRRVNQF. | (SEQ ID NO 123) |

### EXAMPLE 3

### Anti-IL-9 Immunogens

Immunogens for raising an active immune response against IL-9 in mammals and humans are known in the art. Richard et al., PNAS, Jan. 18, 2000, Vol. 97, No. 2, 767-772, discloses anti IL-9 immunogenic compositions and methods for making and administering them (see also U.S. Pat. No. 6,645,486). The anti-IL-9 immunogens disclosed in Richard et al., PNAS, Jan. 18, 2000, Vol. 97, No. 2, 767-772, are useful in the methods of the present invention and the disclosure of Richard et al., PNAS, Jan. 18, 2000, Vol. 97, No. 2, 767-772, is hereby incorporated by reference in its entirety.

### EXAMPLE 4

### Anti-IL-13 Immunogens

Immunogens for raising an active immune response against IL-13 in mammals and humans are known in the art. WO 02/070711 discloses anti IL-13 immunogenic compositions and methods for making and administering them. The anti-IL-13 immunogens disclosed in WO 02/070711 are useful in the methods of the present invention and the disclosure of WO 02/070711 is hereby incorporated by reference in its entirety.

### EXAMPLE 5

### Anti-IL-4 Immunogens

Immunogens for raising an active immune response against IL-4 in mammals and humans are known in the art. WO 02/070711 discloses anti L-4 immunogenic compositions and methods for making and administering them. The anti-IL-4 immunogens disclosed in WO 02/070711 are useful in the methods of the present invention and the disclosure of WO 02/070711 is hereby incorporated by reference in its entirety.

### EXAMPLE 6

### Anti-Eotaxin-2 Immunogens

The amino acid sequence of Eotaxin-2 is known, see Grzegorewski et al., (2001) Cytokine 13; 209-219: and Mayer and Stone (2000) Biochemistry, 39, 8382-8395. The peptide fragments VVIPSPSSMF (SEQ ID NO 124), MFFVSKRIPE, VSKRIPENRV(SEQ ID NO 126), SYQLSSRSTSLK (SEQ ID NO 133), SYQLSSRSTTLK (SEQ ID NO 134) and SYQLSSRSTALK (SEQ ID NO 135) may be used to construct anti-eotaxin-2 immunogens according to the invention.

### EXAMPLE 7

### Anti-Eotaxin-3 Immunogens

The amino acid sequence of Eotaxin-3 is disclosed in Mayer and Stone, (2003) Proteins 50: 184-191. The peptide fragments SDISKTSSFQ (SEQ ID NO 127), FQYSHKPLPW (SEQ ID NO 128), SHKPLPWTWV (SEQ ID NO 129), SYEFTSNSSSQE (SEQ ID NO 136), SYEFTSNSTSQE (SEQ ID NO 137) and SYEFTSNSASQE (SEQ ID NO 138) may be used to construct anti-eotaxin-3 immunogens according to the invention.

### EXAMPLE 8

### Bivalent Anti-Eotaxin and Anti-IL-5 Immunogens

Immunogens for raising an active immune response to both eotaxin and IL-5 may be constructed according to the methods of the invention and use to treat conditions of eosinophilia through the administration of one immunogenic therapeutic vaccine composition. In a particular embodiment of the invention this bivalent immunogenic composition contains antigenic peptide determinants specific to eotaxin epitopes and IL-5 epitopes as haptens conjugated to the same immunogenic protein carrier molecule.

Antigenic peptide determinants for use as the eotaxin specific epitopes may be selected from any of the peptides in SEQ ID NOs. 1-38, 117-119 and 42-61 or any combination thereof.

Antigenic peptide determinants which may be useful as IL-5 epitope haptens include:

| | |
|---|---|
| IPTEIPT; | (SEQ ID NO 62) |
| IPTEIPTS; | (SEQ ID NO 63) |
| IPTEIPTSA; | (SEQ ID NO 64) |
| IPTEIPTSAL; | (SEQ ID NO 65) |
| IPTEIPTSALV; | (SEQ ID NO 66) |
| IPTEIPTSALVK; | (SEQ ID NO 67) |
| IPTEIPTSALVKE; | (SEQ ID NO 68) |
| IPTEIPTSALVKET; | (SEQ ID NO 69) |
| IPTEIPTSALVKETL; | (SEQ ID NO 70) |
| IPTEIPTSALVKETLA; | (SEQ ID NO 71) |
| IPTEIPTSALVKETLAL; | (SEQ ID NO 72) |
| IPTEIPTSALVKETLALL; | (SEQ ID NO 73) |
| IPTEIPTSALVKETLALLS; | (SEQ ID NO 74) |
| IPTEIPTSALVKETLALLST; | (SEQ ID NO 75) |
| IPTEIPTSALVKETLALLSTH; | (SEQ ID NO 76) |
| IPTEIPTSALVKETLALLSTHR; | (SEQ ID NO 77) |
| IPTEIPTSALVKETLALLSTHRT; | (SEQ ID NO 78) |
| IPTEIPTSALVKETLALLSTHRTL; | (SEQ ID NO 79) |
| IPTEIPTSALVKETLALLSTHRTLL; | (SEQ ID NO 80) |
| IPTEIPTSALVKETLALLSTHRTLLI; | (SEQ ID NO 81) |
| IPTEIPTSALVKETLALLSTHRTLLIA; | (SEQ ID NO 82) |
| IPTEIPTSALVKETLALLSTHRTLLIAN; | (SEQ ID NO 83) |
| IPTEIPTSALVKETLALLSTHRTLLIANE; | (SEQ ID NO 84) |
| IPTEIPTSALVKETLALLSTHRTLLIANET; | (SEQ ID NO 85) |
| IPTEIPTSALVKETLALLSTHRTLLIANETL; | (SEQ ID NO 86) |
| IPTEIPTSALVKETLALLSTHRTLLIANETLR; | (SEQ ID NO 87) |
| IPTEIPTSALVKETLALLSTHRTLLIANETLRI; | (SEQ ID NO 88) |
| IPTEIPTSALVKETLALLSTHRTLLIANETLRI | (SEQ ID NO 89) |
| P; | |
| IPTEIPTSALVKETLALLSTHRTLLIANETLRIP | (SEQ ID NO 90) |
| V; | |
| QLCTEEIFQGIGTLESQTV; | (SEQ ID NO 91) |
| LCTEEIFQGIGTLESQTV; | (SEQ ID NO 92) |
| CTEEIFQGIGTLESQTV; | (SEQ ID NO 93) |
| CTEEIFQGIGTLESQT; | (SEQ ID NO 94) |
| CTEEIFQGIGTLESQ; | (SEQ ID NO 95) |
| CTEEIFQGIGTLES; | (SEQ ID NO 96) |
| TVERLFKNLSLIKKYIDGQKKK; | (SEQ ID NO 97) |
| VERLFKNLSLIKKYIDGQKKK; | (SEQ ID NO 98) |
| VERLFKNLSLIKKYIDGQKK; | (SEQ ID NO 99) |
| RVNQFLDYLQEFLGVMNTEWIIES; | (SEQ ID NO 100) |
| VNQFLDYLQEFLGVMNTEWIIES; | (SEQ ID NO 101) |
| PVHKNHQLCTEEIFQGIGTLESQTVQGGTV; | (SEQ ID NO 102) |
| ERLFKNLSLIKKYIDGQKKKCGEERRRVNQ; | (SEQ ID NO 103) |
| FLDYLQEFLGVMNTEWIIES; | (SEQ ID NO 104) |
| RIPVPVHKNHQLC; | (SEQ ID NO 105) |
| QTVQGGT; | (SEQ ID NO 106) |
| KCGEERRR; | (SEQ ID NO 107) |
| TEEIFQG; | (SEQ ID NO 108) |
| EWIIES. | (SEQ ID NO 109) |
| and | |
| SEQ ID NOs 122-123 | |

The antigenic determinants of SEQ ID NOs 62-109 may be coupled to a spacer peptide sequence such as those of SEQ ID NOs. 3941 on either their amino terminal or carboxy terminal end and coupled to the immunogenic carrier such as DT or TT throught the spacer peptide linker. The native sequence of the IL-5 fragment corresponding to the antigenic peptide determinant may also be modified or altered to create an immunomimic useful in the construction of anti-IL-5 immunogenic epitopes. In a specific embodiment of the invention cysteine residues are converted to threonine, although cysteine substitution by serine or alanine instead of threonine could also be used). Examples of such modified peptide sequences are:

| | |
|---|---|
| PVHKNHQLT; | (SEQ ID NO 110) |
| KKYIDGQKKKT; | (SEQ ID NO 111) |
| KKKTGEER; | (SEQ ID NO 112) |
| TGEERRRVNQ; | (SEQ ID NO 113) |
| PVHKNHQLTLPPPPC; | (SEQ ID NO 114) |
| KKYIDGQKKKTSSPPPPC; | (SEQ ID NO 115) |
| and | |
| CPPPPLKKKTGEER. | (SEQ ID NO 116) |

The bivalent immunogens may contain a plurality of antigenic peptide determinants in any combination of eotaxin and IL-5 epitopes conjugated to an immunogenic carrier such as a toxoid peptide. For example a bivalent immunogen useful for raising an active immune response in a human subject against eotaxin and IL-5 may be constructed by conjugating a plurality of antigenic peptide determinants of SEQ ID NOs 56-61 and 114-116, 118, 119, 122 and 123 to an immunogenic carrier such as DT.

### EXAMPLE 9

### Conjugation of Peptide Antigenic Determinants to Immunogenic Carriers

The methods of conjugating the peptide to the immunogenic protein carrier are well known in the art (see for example WO 02/066056). For example the threonine-substituted eotaxin epitope fragments: SGKTPQKAVISSPPPPC (SEQ ID NO 56), FKTKLAKDITSSPPPPC (SEQ ID NO 58), ADPKKKWVQDSSPPPPC (SEQ ID NO 60), may be conjugated to DT or TT carrier proteins via heterobifunctional cross-linking agents. One or more of these eotaxin fragments is cross-linked to DT or TT by reaction with a heterobifunctional cross-linking agent such as N-(epsilon-Maleimidocaproyloxy)-succinimide ester (EMCS) or its water-soluble analogue sulfo-EMCS. In this embodiment, DT or TT are first reacted with the heterobifunctional cross-linking agent via the succinimidyl ester at amino groups on the toxoid. This reaction is preferably accomplished at pH 6.5±0.3 over approximately 1 to 3 hours at room temperature. A ratio of maleimidyl groups to carrier protein of, for example 5:1, 10:1, 15:1 is achieved by reacting the toxoid (DT or other carrier protein) with an appropriate excess of the cross-linking agent. The actual mole excess of cross-linker is determined by titration. The moles of maleimide incorporated per mole of toxoid during titration can be determined by subsequent reaction of the maleimidyl-toxoid with a sulfhydryl compound such as cysteine or beta-mercaptoethanol. The amount of sulfhydryl compound reacted with the maleimidyl-toxoid is most readily determined indirectly by reaction of residual sulfhydryl compound with bis-dithio-nitrobenzoate. After removal of excess cross-linking agent by either diafiltartion or gel permeation chromatography the maleimidyl-toxoid is reacted via its terminal sulfhydryl group with a 1:1 mole excess of eotaxin-spacer peptide over maleimidyl moieties of the activated-toxoid. This conjugation of the peptide to the toxoid is preferably accomplished at pH 6.0±0.3 over approximately 3 to 6 hours at room temperature. Alternatively, this conjugation reaction of the peptide to the maleimidyl-toxoid can be accomplished by overnight reaction at room temperature. It may be preferable to conduct the conjugation reactions with cross-linking agents containing maleimide groups in a vessel. After reaction with peptide the excess of peptide is removed by either diafiltration or gel permeation chromatography into phosphate buffered saline pH 7.2±0.2.

### EXAMPLE 10

### Multivalent Anti-Chemokine Immunogens

Immunogens according to the invention can be constructed by conjugating any number of different peptide epitopes as set forth above in Examples 1-8, SEQ ID NOs. 1-129 in various different combinations using the conjugation methods of Example 9 or by other methods known in the art. In this manner an immunogen can be constructed targeting any combination of target chemokines and in particular Th-2 chemokines selected from the group consisting of eotaxin-1, eotaxin-2, eotaxin-3, IL-4, IL-5, IL-9, or IL-13 and combinations thereof. For example multivalent immunogens may be constructed to a) eotaxin-1, eotaxin-2, eotaxin-3 orb) eotaxin, IL-5, IL-13 or any other combination of Th-2 chemokines. The specific target chemokines selected for construction of the immunogen may vary with the type of immune disorder that is to be treated and its associated cytokine profile.

### EXAMPLE 11

### Formulation of Immunogenic Vaccine Compositions

Formulations suitable for immunogenic presentation of the antigenic determinant toxoid conjugates include, but are not limited to, adsorption to aluminum or alhydrogels, inclusion within liposomes, microsomes or similar microspheres, including microparticulates and nanoparticulates, oil-in-water or water-in-oil emulsions, including multiphasic emulsions, and microemulsions (see for example WO 02/066056). Other potentially suitable formulations include preparations with block-copolymers having adjuvant qualities capable of stimulating the immune response to included antigens, in this case the preferred eotaxin-specific toxoid conjugates. The conjugate immunogens of the invention may be formulated with adjuvants or other immunostimulatory agents in a pharmaceutically acceptable vehicle with components and using methods well known in the art, (see: Vaccine Design, The Subunit and Adjuvant Approach, (1995) Powell and Newman Eds., Plenum Press (New York), Aucouturier et al., Vaccine 19 (2001) 2666-2672).

A specific embodiment of the present invention includes the formulation of the antigenic determinant peptide toxoid conjugates into the aqueous phase of water-in-oil emulsions. A sterile-filtered (0.1 to 0.2 μm) aqueous solution of the peptide toxoid conjugates is combined with a suitable sterile-filtered (0.2 μm) oil mixture containing emulsifiers sufficient to provide for a stable water-in-oil emulsion upon homogenization of the water oil mixture. The emulsification process is practiced as an aseptic procedure within a laminar flow hood or suitable sterile isolator useful for the practice of aseptic formulation and filling of pharmaceutical formulations, including sterile emulsions, that cannot otherwise be sterilized by terminal filtration, heat sterilization or irradiation.

The water-to-oil mixture may be varied in the range 50:50 to 10:90, but more preferably in the range 40:60 to 20:80 water-to-oil. Suitable oil/emulsifier mixtures for this purpose of producing the preferred water-in-oil emulsion may be obtained from the Montanide product range available from SEPPIC, SA, Paris, France. In addition, it may be desirable to further incorporate during the emulsification process a water-soluble adjuvant into the aqueous phase of the final emulsion (Adams, A. Synthetic Adjuvants. 1985 John Wiley & Sons, New York). Examples of suitable adjuvants include, Quill A, QS21, ormuramyl dipepetide (nor-MDP).

Methods of Treating Inflammatory Conditions

The immunogens of the invention may also be administered in treatment regimens with other pharmaceuticals or anti-inflammatory agents. For example in the case of asthma or atopic chronic allergic disorders the patient may be actively immunized with an anti-eotaxin vaccine of the invention so as to control and down-regulate eotaxin levels and the accumulation of eosinophils in the affected tissues while at the same time a rescue medication or anti-asthma or anti-allergy agent is administered in response to an acute attack brought on for example by an overwhelming allergic stimulus. Such additional agents useful in combination treatments may include corticosteroids, cromoglyate, anti-inflammatories, COX-2 inhibitors, leukotriene (receptor) antagonists, xanthines, antihistamines and bronchodilators.

DNA Vaccines

In an alternative embodiment of the invention DNA constructs comprising nucleic acid sequences encoding the eotaxin peptide fragments or other cytokine antigenic determinants described above and further comprising nucleic acid sequences encoding T helper cell epitopes are used as DNA vaccines. Methods of constructing, formulating and administering such DNA vaccines are known in the art and adapted to the chemokine peptide epitopes of the invention, see WO 00/65058, WO 98/31398, Donnelly et al., 1997, Annu. Rev. Immunol. 15: 617-648 and Donnelly et al., 1997, Life Sciences 60: 163-172.

### INCORPORATION BY REFERENCE

Throughout this application reference is made to various publications and patent documents. The disclosures of each of these references, publications and patents, is hereby incorporated by reference into this disclosure in its entirety as part of the description of this application as are the disclosures of the publications and patents that are in turn cited therein.

1. A method for treating a subject for an inflammatory condition which results from eosinophil accumulation which comprises generating an active immune response in the patient comprising autoantibodies to eotaxin and IL-5.

2. A method of treating a subject for a condition which results from asthma, allergy or allergic disease comprising generating an immune response in the subject to eotaxin and IL-5.

3. An immunogenic composition comprising eotaxin or a peptide fragment thereof and IL-5 or a peptide fragment thereof coupled to an immunogenic carrier.

4. An immunogenic composition comprising a T-cell epitope, an epitope derived from IL-5 and an epitope derived from eotaxin.

5. An immunogenic composition according to claim 3 for the treatment of asthma, allergy or allergic disease comprising eotaxin or a portion thereof and IL-5 or a portion thereof conjugated to an immunogenic protein carrier.

6. A method of producing the composition of claim 3 which comprises coupling the eotaxin or a portion thereof and the IL-5 or a portion thereof to the immunogenic carrier.

7. A method of treating a disorder involving an immunomodulatory pathway comprising multiple cytokines the method comprising actively immunizing a subject against two or more cytokines in the pathway so as to elicit autoantibodies in the subject which can bind to and modulate the activities of the cytokines.

8. The method of claim 7 wherein the pathway is the Th-2 pathway.

9. The method of claim 8 wherein the subject is actively immunized against IL-5 and eotaxin.

10. A method for treating a subject for a condition mediated by eotaxin, which comprises generating an active immune response in the subject to eotaxin and at least one cytokine selected from the group consisting of eotaxin-2, eotaxin-3, IL-4, IL-5, IL-9 or IL-13.

11. The method of claim 10 wherein the condition mediated by eotaxin is asthma, allergy or allergic disease.

12. The immunogenic composition of claim 5 comprising at least one peptide sequence selected from the peptide sequences set forth in SEQ ID NOs 1-38, 42-61 and 117-121 and 130-132 and at least one peptide sequence selected from the peptide sequences set forth in SEQ ID NOs 62-116 and 122-123.

13. A method of ameliorating eosinophilia in a human subject which comprises downregulating eotaxin and concurrently down regulating a cytokine selected from the group consisting of eotaxin-2, eotaxin-3,IL-4, IL-5, IL-9, or IL-13.

14. A multivalent immunogenic composition for actively immunizing a subject to treat a Th-2 immune disorder comprising an immunogenic carrier conjugated to a plurality of peptide epitopes derived from two or more Th-2 cytokines.

15. The composition of claim 14 wherein the Th-2 cytokines are selected from the group consisting of eotaxin-1, eotaxin-2, eotaxin-3, IL-4, IL-5, IL-9 or IL-13.
